(19) United States
(12) Patent Application Publication
(10) Pub. No.: US 2020/0078323 A1
(43) Pub. Date: Mar. 12, 2020

(54) METHOD OF TREATING OVARIAN CANCER USING ZETA-STAT

(71) Applicants: Mildred ACEVEDO-DUNCAN, Plant City, FL (US); Tracess SMALLEY, Tampa, FL (US); Rekha PATEL, Tampa, FL (US)

(72) Inventors: Mildred ACEVEDO-DUNCAN, Plant City, FL (US); Tracess SMALLEY, Tampa, FL (US); Rekha PATEL, Tampa, FL (US)

(73) Assignee: University of South Florida, Tampa, FL (US)

(21) Appl. No.: 16/568,476

(22) Filed: Sep. 12, 2019

Related U.S. Application Data
(60) Provisional application No. 62/730,138, filed on Sep. 12, 2018.

(51) Int. Cl. A61K 31/185; A61P 35/00; A61K 9/00

(52) U.S. Cl. CPC A61K 31/185; A61P 35/00; A61K 9/006; A61K 9/007; A61K 9/0034; A61K 9/0019; A61K 9/0031; A61K 9/0043; A61K 9/0056

(57) ABSTRACT

A method of inhibiting the growth or proliferation of ovarian cancer cells is provided. The method comprises contacting the ovarian cancer cells with an effective amount of 8-hydroxynaphthalene-1,3,6-trisulfonic acid (ζ-Stat), a salt of ζ-Stat, a derivative of ζ-Stat, or a salt of a derivative of ζ-Stat. Also provided is a method of treating an ovarian cancer or inhibiting ovarian cancer cell growth or proliferation in a subject by administering to the subject a therapeutically effective amount of ζ-Stat, a salt of ζ-Stat, a derivative of ζ-Stat, or a salt of a derivative of ζ-Stat.

### CROSS-REFERENCE TO RELATED APPLICATION

This application claims the benefit of U.S. Provisional Application Ser. No. 62/730,138, filed Sep. 12, 2018, which is hereby incorporated by reference in its entirety including any tables, figures, or drawings.

### BACKGROUND OF THE INVENTION

Ovarian cancer is the fifth leading cause of cancer deaths and incidence. It is considered the most lethal gynecological cancer. Therefore, therapies to manage and/or treat ovarian cancer are desired.

### BRIEF SUMMARY OF THE INVENTION

Certain embodiments of the invention provide a method of treating an ovarian cancer in a subject. The method comprises administering to the subject a therapeutically effective amount of ζ-Stat or a salt thereof or a derivative of ζ-Stat or a salt thereof. A therapeutically effective amount of ζ-Stat or a salt thereof or a derivative of ζ-Stat or a salt thereof produces in the subject or in or around the cancer cells of the subject a concentration of ζ-Stat or the derivative of ζ-Stat of at least 3 μM.

### BRIEF DESCRIPTION OF THE DRAWINGS

FIGS. 1A-1C. Proliferation curves for TOV21G cells treated with ζ-Stat. TOV21G cells were treated with ζ-Stat for 24, 48, and 72 hours. The control cells were treated with DMSO. (FIG. 1A) Treatments counted on a cellometer. (FIG. 1B) Standard curve for TOV21G cells in WST-1 reagent. (FIG. 1C) Treatments after 72 hours measured using WST-1 reagent.

FIGS. 2A-2C. Proliferation curves for ES-2 cells treated with ζ-Stat. ES-2 cells were treated with ζ-Stat for 24, 48, and 72 hours. The control cells were treated with DMSO. (FIG. 2A) Treatments counted on a cellometer. (FIG. 2B) Standard curve for ES-2 cells in WST-1 reagent. (FIG. 2C) Treatments after 72 hours measured using WST-1 reagent.

FIGS. 3A-3B. Protein expression of PKC-ζ and PKC-ζ of TOV21G and ES-2 cells when treated for 24, 48, and 72 hours with DMSO, ICA-1, and ζ-Stat. (FIG. 3A) TOV21G protein expression levels with treatments and quantified N=3. (FIG. 3B) ES-2 protein expression levels with treatments and quantified N=3.

FIGS. 4A-4B. Protein expression of PKC-ζ and RhoA of TOV21G and ES-2 cells when treated for 72 hours with DMSO and ζ-Stat and genomic expression of RhoA in TOV21G. (FIG. 4A) TOV21G and ES-2 protein expression after 72 hours of treatment. (FIG. 4B) TOV21G genomic expression of RhoA with ζ-Stat treatment after 72 hours of treatment.

FIGS. 5A-5C. In-vivo TOV21G xenographs treated with 1×DPBS (vehicle, N=6) and 20 mg/kg ζ-Stat (N=6) for 30 days. (FIG. 5A) Tumor growth (mm³) for the control mice and the treatment mice. The graph at the bottom represents the average body weight of the mice throughout the treatment period. (FIG. 5B) Tumors extracted at the endpoint of the experiment. The graph below represents the size of tumors from the treatment group represented as the percentage of the control. (FIG. 5C) Enzyme panel tested after the endpoint of the experiment. The bars represent glucose, BUN, ALT, AST and ALKP levels.

FIG. 6. Proposed mechanism for the control of PKC-ζ and RhoA expression in the presence and the absence of ζ-Stat.

### DETAILED DISCLOSURE OF THE INVENTION

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description or the claims, such terms are intended to be inclusive in a manner similar to the term “comprising.” The transitional terms/phrases (and any grammatical variations thereof) “comprising,”, “comprises,” “comprise,” “consisting essentially of,” “consists essentially of,” “consisting,” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Typically, “about” means within a range of 0 to 10% of a given value.

When ranges are used herein, such as for dose ranges, combinations and subcombinations of ranges (e.g., subranges within the disclosed range), specific embodiments therein are intended to be explicitly included.

“Treatment” or “treating” (and grammatical variants of these terms) refer to an approach for obtaining beneficial or desired results including but not limited to therapeutic benefit. A therapeutic benefit is achieved with the eradication or amelioration of one or more of the physiological symptoms associated with the ovarian cancer such that an improvement is observed in the subject, notwithstanding that the subject may still be afflicted with the underlying ovarian cancer.

The term “effective amount” or “therapeutically effective amount” refers to that amount of ζ-Stat, a salt thereof, a derivative of ζ-Stat, or a salt thereof that is sufficient to affect the intended application including but not limited to disease treatment. The therapeutically effective amount may vary depending upon the intended application, the subject, or the disease condition being treated, e.g., the weight and age of the subject, the severity of the cancer, the manner of administration and the like, which can readily be determined by one of ordinary skill in the art. The term also applies to a dose that will induce a particular response in target cells, e.g., reduction in growth or proliferation of target cells. The specific dose will vary depending on the particular compounds chosen, the dosing regimen to be followed, whether it is administered in combination with other compounds, timing of administration, the tissue to which it is administered, and the physical delivery system in which it is carried.

The term “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with ζ-Stat, a salt thereof, a derivative of ζ-Stat, or a salt thereof, its use in the therapeutic compositions of the invention is contemplated. Supplementary active ingredients can also be incorporated into the compositions.

“Subject” refers to an animal, such as a mammal, for example a human. The methods described herein can be useful in both human therapeutics and veterinary applications. Non-limiting examples of subjects include canine, feline, rodent, bovine, equine, and primates.

Ovarian cancer is the most lethal gynecological cancer. Ovarian cancer does not have an efficient screening process and the average stage of diagnosis is stage III. The most common ovarian cancer diagnosis is epithelial ovarian carcinomas, which constitute for 85-90% of prognosis. Of epithelial ovarian carcinomas, clear cell ovarian carcinoma (CCOC) represents 5% of incidence. CCOC presents unique pathological features and has a high reoccurrence rate after treatment. An overexpression of atypical PKCs (PKC-ζ and PKC-ι/λ) has been observed in various malignant cell lines and is linked to pathways for cellular proliferation and invasion. Ovarian cell lines (TOV21G and ES-2) were treated with the atypical PKC-ζ inhibitor ζ-Stat (8-hydroxynaphthalene-1,3,6-trisulfonic acid) and assayed to determine the effects on proliferation and cellular invasion. Mouse xenograph experiments were also performed to determine the effects of ζ-Stat on TOV21G tumor growth in vivo to show that ζ-Stat decreased the proliferation of CCOC cells and decreased wound healing. Tumor growth in athymic female mice is decreased when treated with ζ-Stat and the mouse body weight is maintained. Thus, PKC-ζ is identified herein as a novel target in carcinogenesis and inhibition of this protein decreases the rate of proliferation.

Accordingly, certain embodiments of the invention provide a method of inhibiting the growth or proliferation of ovarian cancer cells comprising contacting the cells with an effective amount of ζ-Stat, a salt of ζ-Stat, a derivative of ζ-Stat, or a salt of a derivative of ζ-Stat. The chemical structure of ζ-Stat is provided below:

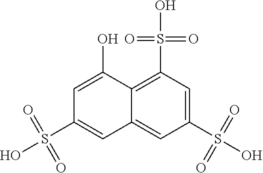 [SMILES: O=S(=O)(O)c1cc(O)c2c(S(=O)(=O)O)cc(S(=O)(=O)O)cc2c1]

The effective amount of ζ-Stat or a salt ζ-Stat can produce a concentration of ζ-Stat of at least about 3 μM, 4 μM, 5 μM, 6 μM, 7 μM, 8 μM, 9 μM or 10 μM in or around the ovarian cancer cells. The effective amount of a derivative of ζ-Stat or a salt of a derivative of ζ-Stat produces a concentration of a derivative of ζ-Stat of at least about 3 μM, 4 μM, 5 μM, 6 μM, 7 μM, 8 μM, 9 μM or 10 μM in or around the ovarian cancer cells.

The derivative of ζ-Stat can contain a substitution at the positions 2, 4, 5, and/or 7 of ζ-Stat with a moiety selected from halo, hydroxyl, amino, amide, thio, cyano, nitro, thioalkyl, carboxylic acid, guanidine, alkyl, alkenyl, alkynyl, alkoxyl, aryl, heteroaryl, cycloalkyl, and heterocycloalkyl, in which the alkyl, alkenyl, alkynyl, alkoxyl, aryl, heteroaryl, cycloalkyl, thioalkyl or heterocycloalkyl moiety is optionally further substituted.

Certain embodiments of the invention provide methods of treating an ovarian cancer in a subject. The method comprises administering to the subject a therapeutically effective amount of ζ-Stat or a salt thereof. A therapeutically effective amount of ζ-Stat or a salt thereof produces in the subject or in or around the cancer cells of the subject a concentration of ζ-Stat of at least 3 μM. In preferred embodiments, the therapeutically effective amount of ζ-Stat or a salt thereof produces in the subject or in or around the cancer cells of the subject a concentration of ζ-Stat of at least about 3 μM, 4 μM, 5 μM, 6 μM, 7 μM, 8 μM, 9 μM or 10 μM. Based on the molecular weight of the ζ-Stat or a salt thereof administered to a subject, a therapeutically effective amount ranges from about 1 mg/kg to about 50 mg/kg.

Certain embodiments of the subject invention provide methods of treating an ovarian cancer in a subject by administering to the subject a therapeutically effective amount of a derivative of ζ-Stat or a salt thereof. A derivative of ζ-Stat can contain a substitution at the positions 2, 4, 5, and/or 7 of ζ-Stat with a moiety selected from halo, hydroxyl, amino, amide, thio, cyano, nitro, thioalkyl, carboxylic acid, guanidine, alkyl, alkenyl, alkynyl, alkoxyl, aryl, heteroaryl, cycloalkyl, and heterocycloalkyl, in which the alkyl, alkenyl, alkynyl, alkoxyl, aryl, heteroaryl, cycloalkyl, thioalkyl or heterocycloalkyl moiety is optionally further substituted. A therapeutically effective amount of a derivative of ζ-Stat or a salt thereof produces in the subject or in or around the cancer cells of the subject a concentration of the derivative of ζ-Stat of at least 3 μM. In preferred embodiments, the therapeutically effective amount of a derivative of ζ-Stat or a salt thereof produces in the subject or in or around the cancer cells of the subject a concentration of a derivative of ζ-Stat of at least about 3 μM, 4 μM, 5 μM, 6 μM, 7 μM, 8 μM, 9 μM or 10 μM. Based on the molecular weight of the derivative of ζ-Stat or a salt thereof administered to a subject, a therapeutically effective amount ranges from about 1 mg/kg to about 50 mg/kg.

Further embodiments of the invention provide methods of reducing growth or proliferation of ovarian cancer cells in a subject. The method comprises administering to the subject a therapeutically effective amount of ζ-Stat or a salt thereof. A therapeutically effective amount of ζ-Stat or a salt thereof produces in the subject or in or around the cancer cells of the subject a concentration of ζ-Stat of at least 3 μM. In preferred embodiments, the therapeutically effective amount of ζ-Stat or a salt thereof produces in the subject or in or around the cancer cells of the subject a concentration of ζ-Stat of at least about 3 μM, 4 μM, 5 μM, 6 μM, 7 μM, 8 μM, 9 μM or 10 μM.

Even further embodiments of the subject invention provide methods of reducing growth or proliferation of ovarian cancer cells in a subject by administering to the subject a therapeutically effective amount of a derivative of ζ-Stat or a salt thereof. A derivative of ζ-Stat can contain a substitution at the positions 2, 4, 5, and/or 7 of ζ-Stat with a moiety selected from halo, hydroxyl, amino, amide, thio, cyano, nitro, thioalkyl, carboxylic acid, guanidine, alkyl, alkenyl, alkynyl, alkoxyl, aryl, heteroaryl, cycloalkyl, and heterocycloalkyl, in which the alkyl, alkenyl, alkynyl, alkoxyl, aryl, heteroaryl, cycloalkyl, thioalkyl or heterocycloalkyl moiety is optionally further substituted. A therapeutically effective amount of a derivative of ζ-Stat or a salt thereof produces in the subject or in or around the cancer cells of the subject a concentration of the derivative of ζ-Stat of at least 3 μM. In preferred embodiments, the therapeutically effective amount of a derivative of ζ-Stat or a salt thereof produces in the subject or in or around the cancer cells of the subject a concentration of a derivative of ζ-Stat of at least about 3 μM, 4 μM, 5 μM, 6 μM, 7 μM, 8 μM, 9 μM or 10 μM.

ζ-Stat or a salt thereof or a derivative of ζ-Stat or a salt thereof can be administered to the subject via an appropriate route that can be readily determined by a person of ordinary skill in the art. Such routes of administration include oral, pulmonary, buccal, suppository, intravenous, intraperitoneal, intranasal, intravaginal, intratumoral, intramuscular or subcutaneous routes. Additional routes of administration are well known to a skilled artisan and such embodiments are within the purview of this invention. A person of ordinary skill in the art can determine an appropriate route of administration based on specific parameters.

### Materials and Methods

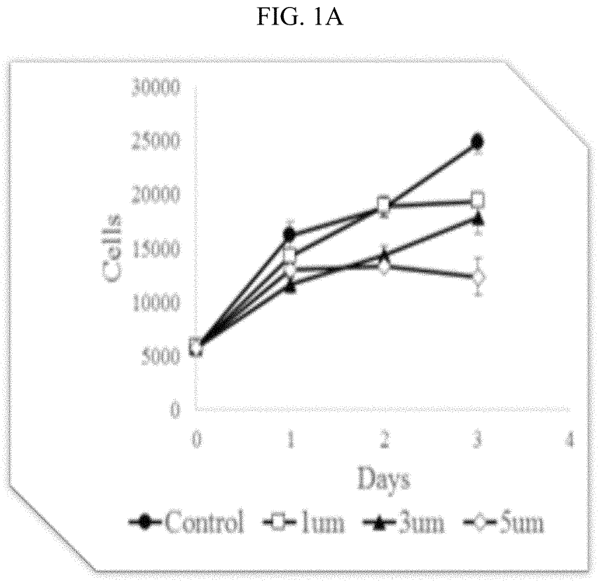

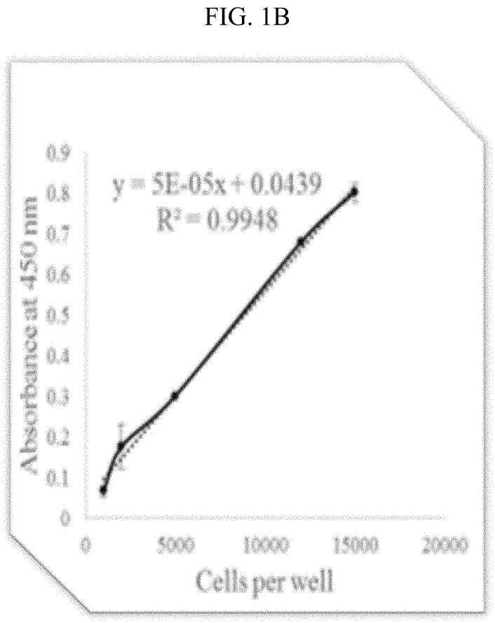

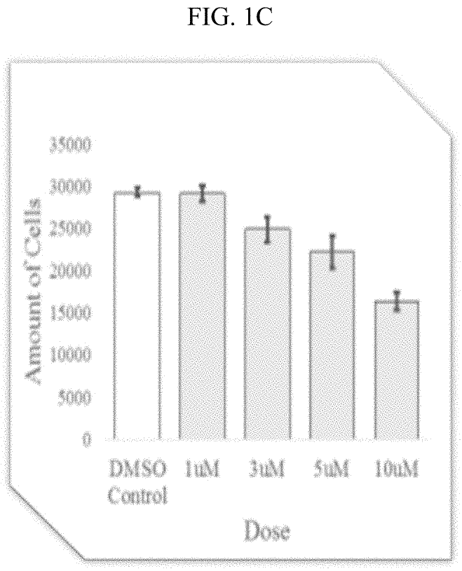

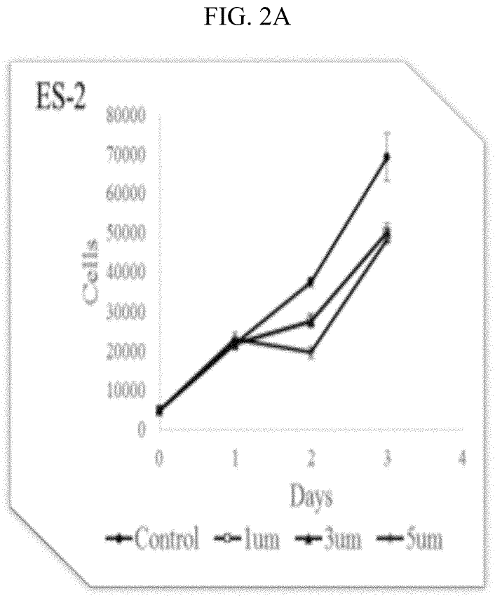

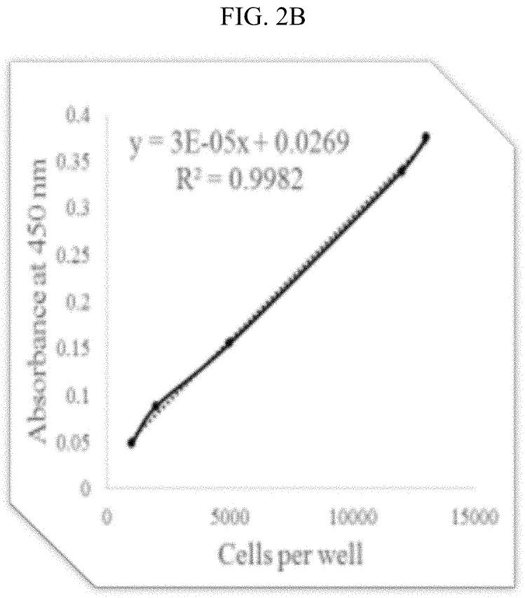

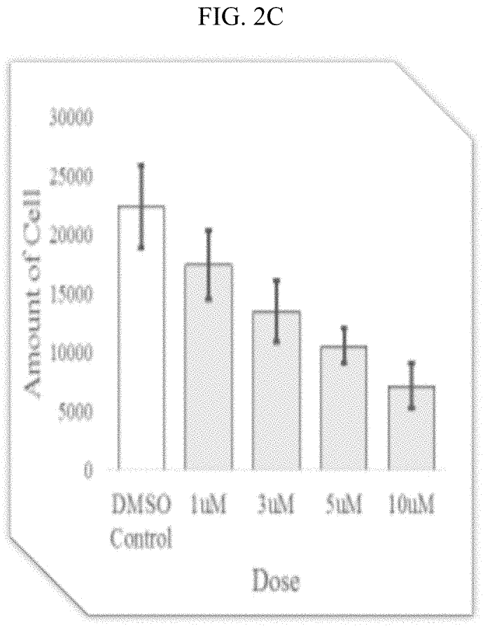

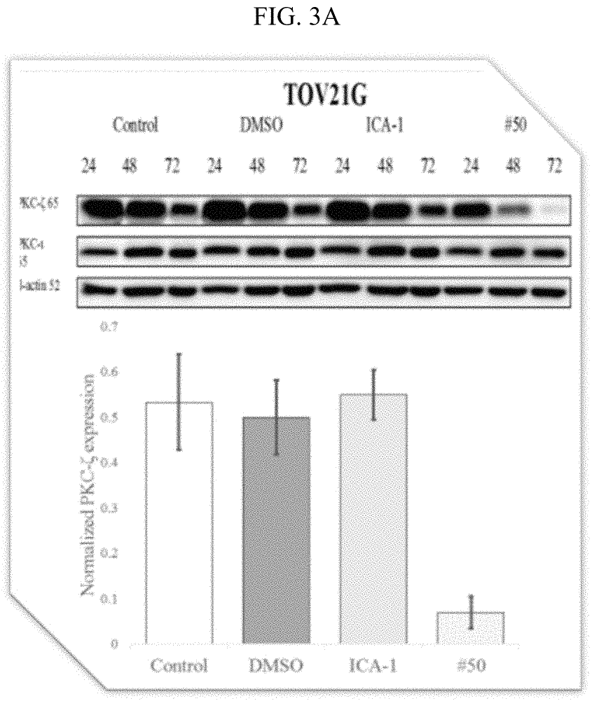

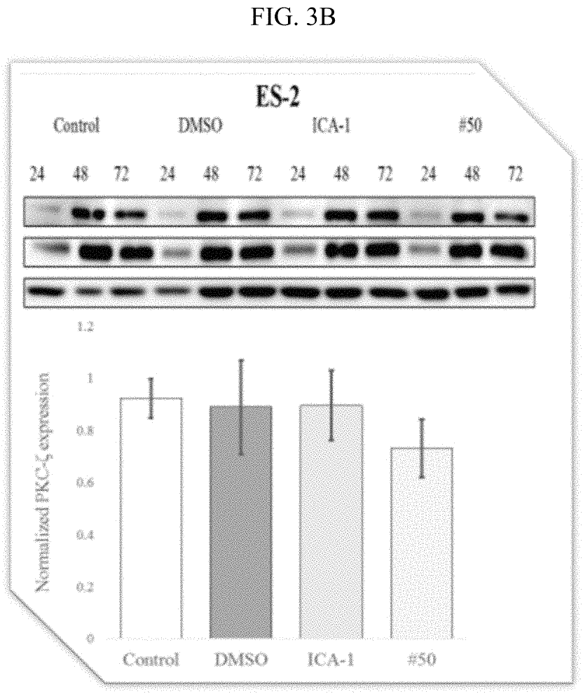

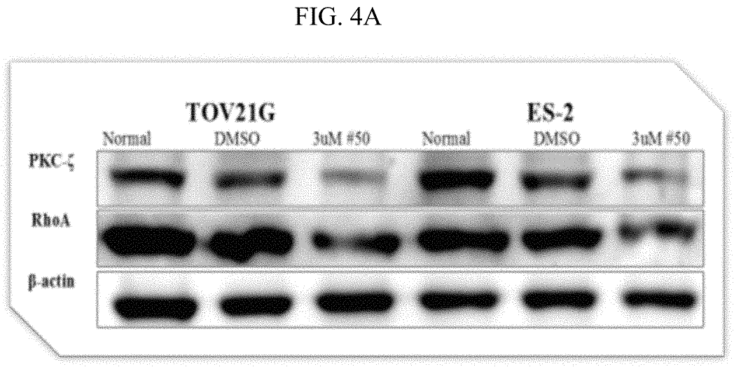

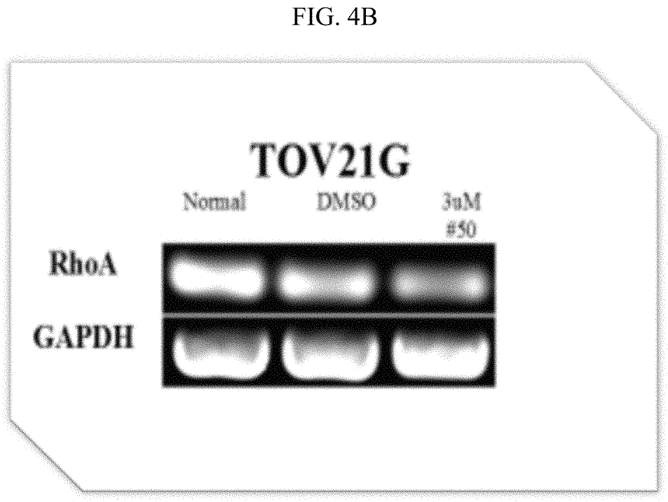

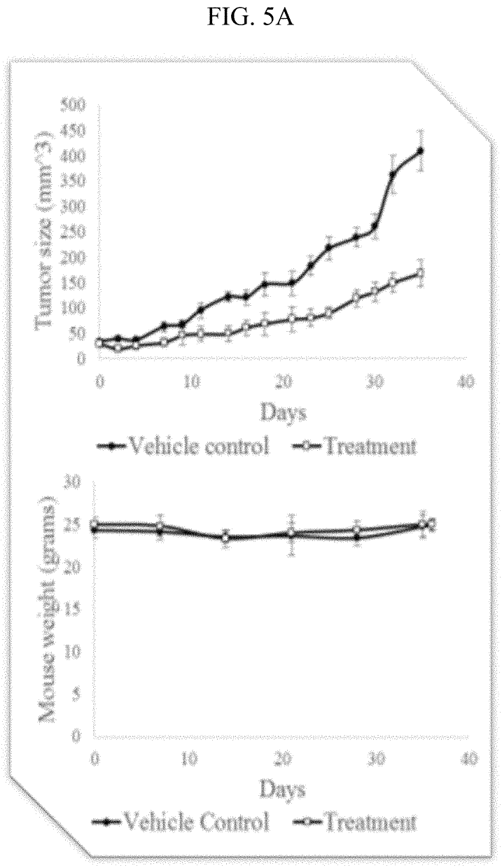

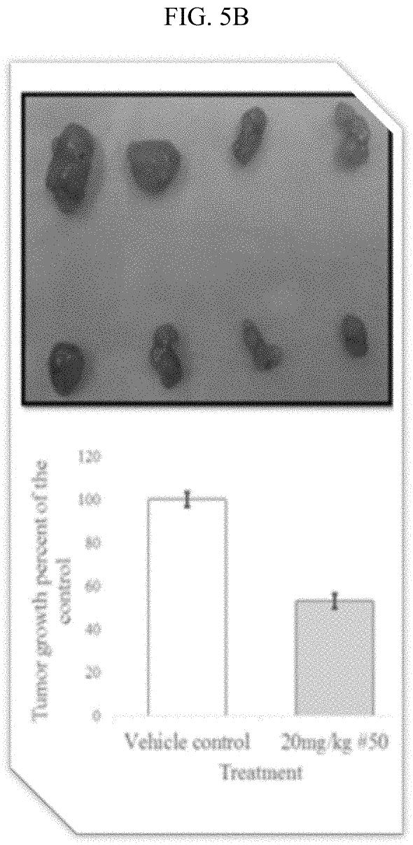

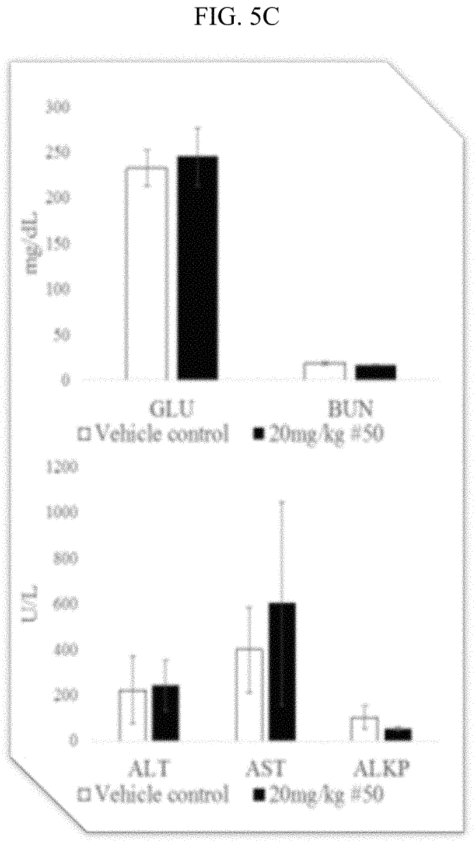

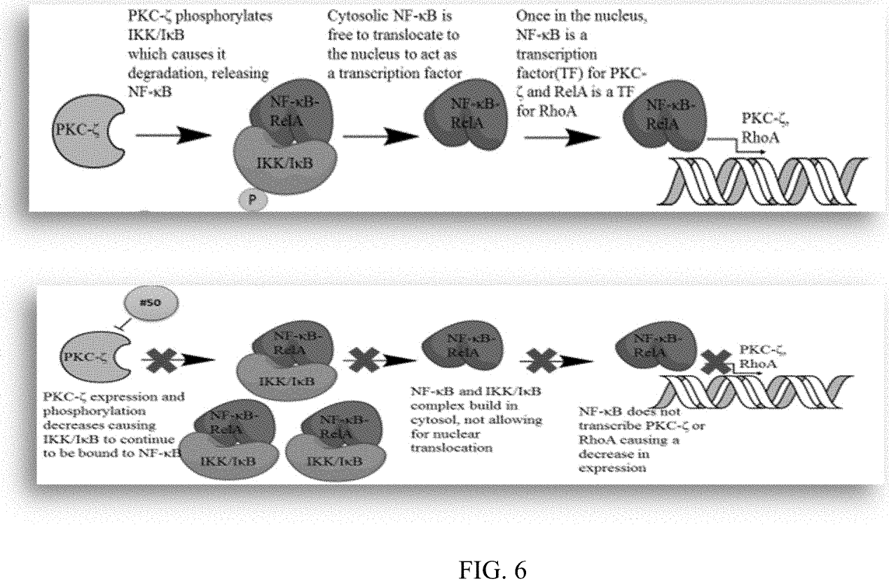

Effects of ζ-Stat on athymic mice ovarian xenograph were tested. Twelve athymic female nude mice weighing between 20-25 g and more than 10 weeks of age were used. The mice were divided into two groups after TOV21G cells (CRL-11730, American Type Culture Collection) were implanted (A million cells/mouse flank in 0.2 ml of media). The first group was the control group (n=6), which received 100 μl of DPBS (21-031-CM, Corning Cellgro, VA). The second group (n=6) received 100 μl of 20 mg/kg of ζ-Stat dissolved in DPBS. The tumor volume was calculated using the formula: length×width×width×½. Three days after implantation of the cells, the tumors were treated as of day 0. The treatments were administered every other day subcutaneously intra-tumor and around the tumor site for 16 days. The results are shown in FIGS. 1A to 5C.

Cellular proliferation studies show that the proliferation of TOV21G and ES-2 cells is decreased when treated with ζ-Stat. Protein and genomic studies show that ζ-Stat knocks down the expression of PKC-ζ but not the expression of PKC-ι, suggesting that it is selective to PKC-ζ. Also, knockdown of PKC-ζ also knocks down the expression of RhoA at the protein level and the genomic level in TOV21G cells. Tumor growth is reduced in mouse xenographs with TOV21G when treated with 20 mg/kg of ζ-Stat. ζ-Stat did not have a toxic effect after 30 days of treatment.

All patents, patent applications, provisional applications, and publications referred to or cited herein are incorporated by reference in their entirety, including all figures and tables, to the extent they are not inconsistent with the explicit teachings of this specification.

It should be understood that embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Siegel, R. L., Miller, K. D., & Jemal, A. (2015). Cancer statistics, 2015. CA: a cancer journal for clinicians, 65(1), 5-29.
2. Hirai, T., & Chida, K. (2003). Protein kinase (PKC): activation mechanisms and cellular functions. Journal of Biochemistry, 133(1), 1-7.
3. Kazanietz, M. (Ed.). (2010). Protein kinase C in cancer signaling and therapy. New York: Springer Science+Business Media, LLC. doi:10.1007/978-1-60761-543-9.
4. Chua H L, Bhat-Nakshatri P, Clare S E, Morimiya A, Badve S, Nakshatri H. NF-kappaB represses E-cadherin expression and enhances epithelial to mesenchymal transition of mammary epithelial cells: potential involvement of ZEB-1 and ZEB-2. Oncogene [Internet]. 2007; 26(5):711-24.
5. Ku, F. C., Wu, R. C., Yang, L. Y., Tang, Y. H., Chang, W. Y., Yang, J. E., . . . Lai, C. H. (2018). Clear cell carcinomas of the ovary have poorer outcomes compared with serous carcinomas: Results from a single-center Taiwanese study. Journal of the Formosan Medical Association, 117(2), 117-125. https://doi.org/10.1016/j fma.2017.03.007.
6. Rimessi, A., Patergnani, S., Ioannidi, E., & Pinton, P. (2013). Chemoresistance and Cancer-Related Inflammation: Two Hallmarks of Cancer Connected by an Atypical Link, PKCζ. Frontiers in Oncology, 3(January 2014). See world-wide-website: doi.org/10.3389/fonc.2013.0023.
7. United States Patent Application Publication Number US 2018/0008564.
8. U.S. Pat. No. 9,301,965.
9. Seto, Kelly K Y, and Irene L. Andrulis. Atypical protein kinase C zeta: potential player in cell survival and cell migration of ovarian cancer. PloS one 10.4 (2015): e0123528.
10. Nazarenko, Irina, et al. “Atypical protein kinase C ζ exhibits a proapoptotic function in ovarian cancer.” Molecular Cancer Research (2010): 1541-7786.
11. Cooke, Mariana, et al. “Protein kinase C in cancer: The top five unanswered questions.” Molecular carcinogenesis 56.6 (2017): 1531-1542.
12. Butler, Amanda, et al. “Protein Kinase C Zeta Regulates Human Pancreatic Cancer Cell Transformed Growth and Invasion through a STAT3-Dependent Mechanism.” PLOS ONE, August 2013, Volume 8, Issue 8, e72061, pp. 1-11.

We claim:

1. A method of inhibiting the growth or proliferation of ovarian cancer cells comprising contacting the ovarian cancer cells with an effective amount of 8-hydroxynaphthalene-1,3,6-trisulfonic acid (ζ-Stat), a salt of ζ-Stat, a derivative of ζ-Stat, or a salt of a derivative of ζ-Stat.

2. The method of claim 1, wherein the effective amount of ζ-Stat or a salt ζ-Stat produces a concentration of ζ-Stat of at least 3 μM in or around the ovarian cancer cells.

3. The method of claim 1, wherein the effective amount of a derivative of ζ-Stat or a salt of a derivative of ζ-Stat produces a concentration of a derivative of ζ-Stat of at least 3 μM in or around the ovarian cancer cells.

4. The method of claim 1, wherein the derivative of ζ-Stat comprises a substitution at the positions 2, 4, 5, and/or 7 of ζ-Stat with a moiety selected from halo, hydroxyl, amino, amide, thio, cyano, nitro, thioalkyl, carboxylic acid, guanidine, alkyl, alkenyl, alkynyl, alkoxyl, aryl, heteroaryl, cycloalkyl, and heterocycloalkyl, in which the alkyl, alkenyl, alkynyl, alkoxyl, aryl, heteroaryl, cycloalkyl, thioalkyl or heterocycloalkyl moiety is optionally further substituted.

5. A method of treating ovarian cancer in a subject or inhibiting ovarian cancer cell proliferation in a subject, the method comprising administering to the subject a therapeutically effective amount of 8-hydroxynaphthalene-1,3,6-trisulfonic acid (ζ-Stat) or a salt thereof.

6. The method of claim 5, wherein the therapeutically effective amount of ζ-Stat or a salt thereof produces in the subject a concentration of ζ-Stat of at least 3 μM.

7. The method of claim 5, wherein the therapeutically effective amount of ζ-Stat or a salt thereof produces in or around the cancer cells of the subject a concentration of ζ-Stat of at least 3 μM.

8. The method claim 5, comprising administering ζ-Stat or a salt thereof or a derivative of ζ-Stat or a salt thereof via oral, pulmonary, buccal, suppository, intravenous, intraperitoneal, intranasal, intravaginal, intratumoral, intramuscular or subcutaneous route.

9. A method of treating ovarian cancer in a subject or inhibiting ovarian cancer cell proliferation in a subject, the method comprising administering to the subject a therapeutically effective amount of a derivative of ζ-Stat or a salt thereof, wherein the derivative of ζ-Stat comprises a substitution at the positions 2, 4, 5, and/or 7 of ζ-Stat with a moiety selected from halo, hydroxyl, amino, amide, thio, cyano, nitro, thioalkyl, carboxylic acid, guanidine, alkyl, alkenyl, alkynyl, alkoxyl, aryl, heteroaryl, cycloalkyl, and heterocycloalkyl, in which the alkyl, alkenyl, alkynyl, alkoxyl, aryl, heteroaryl, cycloalkyl, thioalkyl or heterocycloalkyl moiety is optionally further substituted.

10. The method of claim 9, wherein the therapeutically effective amount of the derivative of ζ-Stat or a salt thereof produces in the subject a concentration of the derivative of ζ-Stat of at least 3 μM.

11. The method of claim 9, wherein the therapeutically effective amount of the derivative of ζ-Stat or a salt thereof produces in or around the cancer cells of the subject a concentration of the derivative of ζ-Stat of at least 3 μM.

12. The method of claim 9, comprising administering ζ-Stat or a salt thereof or a derivative of ζ-Stat or a salt thereof via oral, pulmonary, buccal, suppository, intravenous, intraperitoneal, intranasal, intravaginal, intratumoral, intramuscular or subcutaneous route.
